(19) United States
(12) Patent Application Publication
(10) Pub. No.: US 2010/0191198 A1
(43) Pub. Date: Jul. 29, 2010

(54) Wound Filler Material with Improved Nonadherency Properties

(75) Inventor: David G. Heagle, Taunton, MA (US)

Correspondence Address: TYCO HEALTHCARE GROUP LP, 15 HAMPSHIRE STREET, MANSFIELD, MA, 02048, US

(73) Assignee: TYCO HEALTHCARE GROUP LP, Mansfield, MA (US)

(21) Appl. No.: 12/510,637

(22) Filed: Jul. 28, 2009

Related U.S. Application Data
(60) Provisional application No. 61/147,189, filed on Jan. 26, 2009.

(51) Int. Cl. A61M 1/00; B05D 3/00; B29C 47/00; B32B 37/02; A61F 13/00

(52) U.S. Cl. 604/313; 427/2.31; 264/211.12; 156/244.24

(57) ABSTRACT

An apparatus for promoting the healing of an exuding wound includes a wound cover for defining a reservoir over a wound in which a negative pressure may be maintained. The cover may form a substantially fluid-tight seal around the wound and permit fluid communication between the reservoir and a vacuum source suitable for providing an appropriate negative pressure to the reservoir to stimulate healing of the wound. A wound filler positioned between the wound and the wound cover includes a fibrous material treated with a surface modification additive.

### CROSS-REFERENCE TO RELATED DOCUMENTS

The present invention claims the benefit of and priority to U.S. provisional patent Application Ser. No. 61/147,189, filed on Jan. 26, 2009, disclosure of which may be referred to herein by reference.

### BACKGROUND

1. Technical Field

The present disclosure relates generally to wound dressings, and in particular to a wound filler material with improved nonadherency properties for managing wound exudates in a negative pressure wound therapy (NPWT) apparatus.

2. Background of Related Art

Wound dressings are generally placed over a wound to protect and promote healing of the wound. In the case of exuding wounds, such as pressure sores, ulcers and burns, it is customary to provide a dressing having an absorbent material for absorbing at least a portion of the wound exudate as it is produced. Absorbing exudates promotes healing by removing potentially harmful bacteria from the wound bed, and also facilitates exudates removal from the wound bed via a vacuum system. Removal of excess exudates prevents damage to the surrounding skin that can be caused by an excessively moist environment.

The absorbent material temporarily stores the excess exudates until such time as they may be removed, by means of the vacuum system or as the dressing is periodically replaced with a new dressing. The underlying wound, still in the process of healing and regenerating tissue, may be damaged upon removal of the wound dressing as the dressing can get stuck in dried exudates or other coagulum formed therein. Removing the stuck fibers can be a labor intensive procedure that may be painful and further damage or cause trauma to the wound. Neglecting to remove these stray fibers may cause irritation, increase the risk of infection, and otherwise inhibit natural healing of the wound.

One technique that may utilize a dressing with an absorbent material is known as negative pressure wound therapy (NPWT). The absorbent material may be positioned in a reservoir over the wound where a negative pressure may be maintained. The reservoir subjects the wound to a sub-atmospheric pressure to effectively draw wound fluid, including liquid exudates, from the wound without the continuous use of the vacuum pump. Hence, vacuum pressure may be applied once, or in varying intervals depending on the nature and severity of the wound. This technique has been found to promote blood flow to the area, stimulate the formation of granulation tissue, and encourage the migration of healthy tissue over the wound. An NPWT apparatus may also serve to draw exudates from the absorbent material out of the dressing without requiring that the entire dressing be changed. When an NPWT procedure is complete, the absorbent material must be removed and is thus subject to the difficulties that may be caused by stuck fibers. Accordingly, a non-adherent material suitable for use in wound dressings, including wound dressings adapted for use in advanced wound therapy procedures such as NPWT, would be helpful.

### SUMMARY

The present disclosure describes an apparatus for promoting the healing of an exuding wound. The apparatus includes a wound cover for defining a reservoir over a wound in which a negative pressure may be maintained. The cover may form a substantially fluid-tight seal around the wound and permit fluid communication between the reservoir and a vacuum source suitable for providing an appropriate negative pressure to the reservoir to stimulate healing of the wound. A wound filler positioned between the wound and the wound cover includes a fibrous material treated with a surface modification additive.

In embodiments, the surface modification additive is externally applied to the fibrous material. The surface modification additive may be applied by coating, spraying, dipping, brushing, or melting a chemical composition, solution, or melt containing the surface modification additive onto the surface of the fibrous material. In other embodiments, the surface modification additive is internally applied to the fibrous material by extruding the additive with the raw polymer used to form the fibers of the fibrous material. The surface modification additive may contain a wax, silicone, fluorochemical, or other hydrophobe that improves the nonadherency of the fibrous material. In embodiments, the surface modification additive imparts a critical wetting surface tension that is less than 50 dynes per centimeter. A bioactive agent, such as an antimicrobial, may be combined with the surface modification additive to impart additional characteristics to the wound filler.

Methods of forming the wound filler are also described. In accordance with the methods of the present disclosure, a surface modification additive is topically applied to a fibrous material and cured to form a finish on the fibrous material. In other methods, a raw polymer and a surface modification additive may be separated heated and then combined and extruded to form homogenous fibers. The fibers may then be formed into a fibrous web to form a wound filler.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1 is a cross sectional view of an NPWT apparatus incorporating a wound dressing formed in accordance with the present disclosure;

FIG. 2 is perspective view of a surface modified fibrous material which forms the wound filler of FIG. 1;

FIG. 3A is a perspective view of a fibrous material coated with a surface modification additive in accordance with the present disclosure;

FIG. 3B is a cross-sectional view of a fiber coated with a surface modification additive in accordance with the present disclosure;

FIGS. 4A-4B are schematic views depicting the application of a chemical composition containing a surface modification additive to a fibrous material as described in at least one of the embodiments of the present disclosure;

FIGS. 5A-5B are schematic views depicting the application of a solution containing a surface modification additive to a fibrous material as described in at least one of the embodiments of the present disclosure;

FIGS. 6A-6B are schematic views depicting the application of a film containing a surface modification additive to a fibrous material as described in at least one of the embodiments of the present disclosure; and

FIG. 7 is a schematic view depicting the extrusion of a fiber from a raw polymer and a surface modification additive as described in at least one of the embodiments of the present disclosure.

### DETAILED DESCRIPTION OF THE EMBODIMENTS

The wound dressing of the present disclosure incorporates a surface modified fibrous wound filler suitable for absorbing and/or transferring wound exudates therethrough while exhibiting a low tendency to become attached to a healing wound bed. While the specification refers to the use of the surface modified fibrous material with NPWT, the material may be used in a variety of wound care applications, such as a packing material for low exuding wounds.

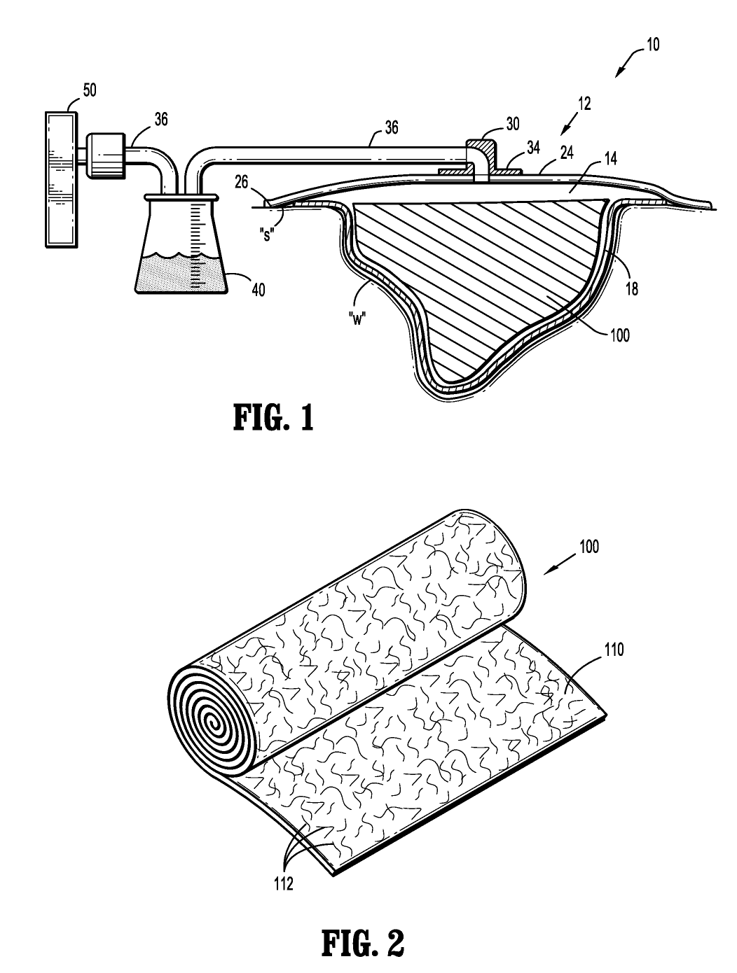

Referring now to the drawings, in which like reference numerals identify identical or substantially similar parts throughout the several views, FIG. 1 depicts an NPWT apparatus according to the present disclosure referred generally as 10 for use on a wound “w” surrounded by healthy skin “s.” The NPWT apparatus 10 includes a wound dressing 12 positioned relative to the wound “w” to define a reservoir 14 in which a negative pressure appropriate to stimulate healing may be maintained.

Wound dressing 12 may include an optional contact layer 18 positioned in direct contact with the bed of wound “w” and may be formed from perforated film material. An appropriate perforated material permits the negative pressure applied to the reservoir to penetrate into the wound “w,” and also permits exudates to be drawn through the contact layer 18. A non-adherent material may be selected such that contact layer 18 does not tend to cling to the wound “w” or surrounding tissue when it is removed. One exemplary material that may be used as a contact layer 18 is sold under the trademark XEROFLO® by Tyco Healthcare Group LP (d/b/a Covidien), or the commercially available CURITY® non-adherent dressing offered by Tyco Healthcare Group LP (d/b/a Covidien). This dressing is an open mesh knitted fabric material made from a cellulose acetate and impregnated with a petrolatum emulsion.

Wound filler 100 is positioned in the wound “w”, over the optional contact layer 18, and is intended to allow wound dressing 12 to transfer wound exudates. Alternatively, wound filler 100 may be positioned in wound “w” without contact layer 18 as the wound filler of the present disclosure is substantially non-adherent and does not tend to cling to the wound “w”. Wound filler 100 is conformable such that it may assume the shape of any wound “w” and may be packed up to the level of healthy skin “s.” The wound filler 100 may be formed in any shape and size. For example, the wound filler 100 may be a preformed shape, such as square or circle sponges, of various sizes. Alternatively, the wound filler 100 may be custom fit by cutting to size. As discussed in greater detail below, the filler 100 may be formed from a fibrous material that has been treated with a surface modification additive. In embodiments, the use of the non-adherent wound filler 100 may eliminate the need for the contact layer 18.

Wound dressing 12 also includes a cover layer 24 in the form of a flexible membrane. Cover layer 24 may be positioned over the wound “w” such that a biocompatible adhesive at the periphery 26 of the cover layer 24 forms a substantially fluid-tight seal with the surrounding skin “s.” Thus, cover layer 24 may act as both a microbial barrier to prevent contaminants from entering the wound “w,” and also a fluid barrier maintaining the integrity of vacuum reservoir 14. Cover layer 24 is preferably formed from a moisture vapor permeable membrane to promote the exchange of oxygen and moisture between the wound “w” and the atmosphere. A membrane that provides a sufficient moisture vapor transmission rate (MVTR) is a transparent membrane sold under the trade name POLYSKIN®II by Tyco Healthcare Group LP (d/b/a Covidien). A transparent membrane permits an assessment of wound conditions to be made without requiring removal of the cover layer 24. Alternatively, cover layer 24 may comprise an impermeable membrane 24. As a further alternative, cover layer 24 may be substantially rigid.

A vacuum port 30 having a flange 34 may also be included in wound dressing 12 to facilitate connection of the wound dressing 12 to fluid conduit 36. Fluid conduit 36 defines a fluid flow path leading through the apparatus 10. The vacuum port 30 may be configured as a rigid or flexible, low-profile component, and may be adapted to receive a vacuum tube 38 in a releasable and fluid-tight manner. An adhesive on the underside of flange 34 may provide a mechanism for affixing the vacuum port 30 to the dressing 12, or alternatively flange 34 may be positioned within reservoir 14 (not shown) such that an adhesive on an upper side of the flange 34 affixes the vacuum port 30. However it is affixed to the dressing, a hollow interior of the vacuum port 30 provides fluid communication between the fluid conduit 36 and the reservoir 14. Vacuum port 30 may be provided as a pre-affixed component of dressing 12, as a component of fluid conduit 36 or entirely independently. Alternatively, vacuum port 30.may be eliminated from dressing 12 if other provisions are made for providing fluid communication with the fluid conduit 36.

Fluid conduit 36 extends from the vacuum port 30 to provide fluid communication between the reservoir 14 and collection canister 40. Any suitable conduit may be used for fluid conduit 36 including those fabricated from flexible elastomeric or polymeric materials. Fluid conduit 36 may connect to the vacuum port 30, the canister 40, or other apparatus components by conventional air tight means such as friction fit, bayonet coupling, or barbed connectors. The conduit connections may be made permanent, or alternatively a quick-disconnect or other releasable means may be used to provide some adjustment flexibility to the apparatus 10.

Collection canister 40 may comprise any container suitable for containing wound fluids. For example, a rigid bottle may be used as shown or alternatively a flexible polymeric pouch may be appropriate. Collection canister 40 may contain an absorbent material to consolidate or contain the wound drainage or debris. For example, super absorbent polymers (SAP), silica gel, sodium polyacrylate, potassium polyacrylamide or related compounds may be provided within canister 40. At least a portion of canister 40 may be transparent to assist in evaluating the color, quality or quantity of wound exudates. A transparent canister may thus assist in determining the remaining capacity of the canister or when the canister should be replaced.

Leading from collection canister 40 is another section of fluid conduit 36 providing fluid communication with vacuum source 50. Vacuum source 50 generates or otherwise provides a negative pressure to the NPWT apparatus 10. Vacuum source 50 may comprise a peristaltic pump, a diaphragmatic pump or other mechanism that draws fluids, e.g. atmospheric gases and wound exudates, from the reservoir 14 appropriate to stimulate healing of the wound “w.” The vacuum source 40 is adapted to produce a sub-atmospheric pressure in the reservoir 14 ranging between about 20 mmHg and about 500 mmHg, in embodiments, from about 75 mmHg to about 125 mmHg, and in other embodiments from about 44 mm to about 80 mm.

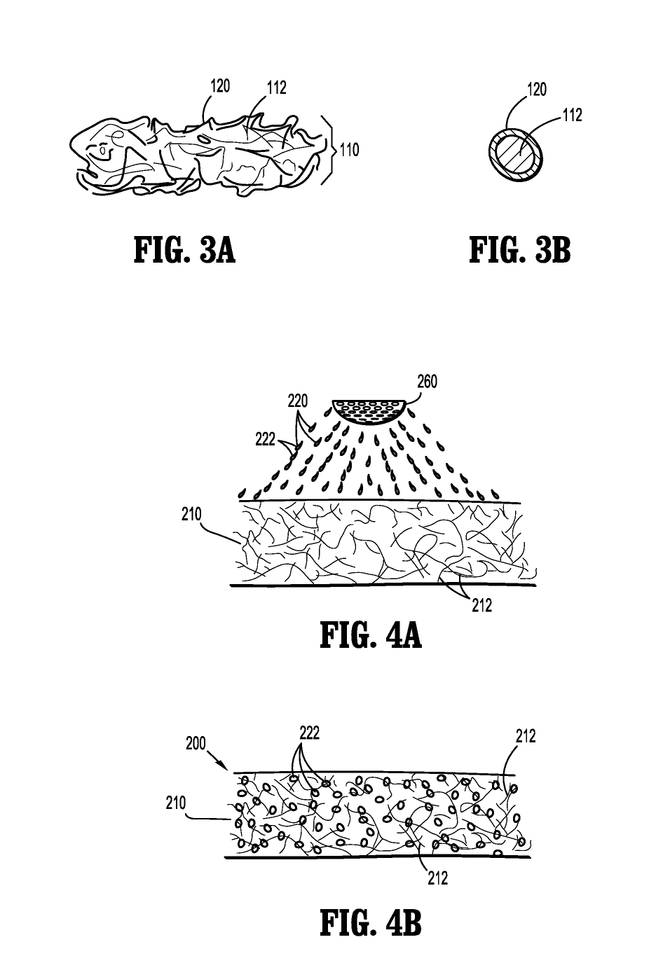

Referring now to FIG. 2, a wound filler 100 may be formed from a fibrous tow or nonwoven material 110 including surface modification additive 120, as illustrated in FIGS. 3A and 3B. The fibrous material 110 may be adapted to absorb wound fluid and exudates, or may be adapted to convey or wick fluids or exudates from the wound bed for removal by vacuum source 40. Fibrous material 110 may be resilient and compressible so that it can easily conform and assume the shape of any wound “w”, such as an irregular-shaped wound bed.

Fibrous material 110 may be a continuous filament fiber or a mass of fibers of a natural, synthetic, or composite material, randomly or systematically arranged and/or coupled together to form a batting having a desirable loft. A non-exhaustive list of materials from which the fibrous material 110 may be fabricated includes, but is not limited to, polymers and polymer blends selected from the group consisting of polyolefins (such as polyethylene and polypropylene including atactic, isotactic, syndiotactic, and blends thereof as well as, polyisobutylene and ethylene-alphaolefins copolymers, and fluorinated polyolefin such as polytetrafluoroethylene); polyesters (such as polyethylene terephthalate and polybutylene terephthalate); acrylic polymers and copolymers; modacrylics; vinyl halide polymers and copolymers (such as polyvinyl chloride); polyvinyl ethers (such as polyvinyl methyl ether and polyvinyl alcohol); polyvinylidene halides (such as polyvinylidene fluoride and polyvinylidene chloride); polyacrylonitrile; polyvinyl ketones; polyvinyl aromatics (such as polystyrene); polyvinyl esters (such as polyvinyl acetate); copolymers of vinyl monomers with each other and olefins (such as etheylene-methyl methacrylate copolymers, acrylonitrile-styrene copolymers, ABS resins and ethylene-vinyl acetate copolymers); polyamides (such as nylon 4, nylon 6, nylon 6.6, nylon 610, nylon 11, nylon 12 and polycaprolactam); alkyd resins; polycarbonates; polyoxymethylenes; polyimides; polyethers; epoxy resins; aramids, polyurethanes; rayon; rayon-triacetate; and spandex.

The fibrous material 110 may be nonwoven and formed by mechanically, chemically, or thermally bonding the fiber(s) 112 of the material. Fibrous material 110 may be formed into a sheet or web. In embodiments, fiber(s) 112 may be mechanically bound by entangling the fiber(s) to form fibrous material 110 by means other than knitting or weaving, such as matting, pressing, needlepunching, or otherwise interlocking the fiber(s) to form a binderless network of fibers. In other embodiments, the fiber(s) 112 of fibrous material 110 may be chemically bound by use of an adhesive, such as a hot melt adhesive, or thermally bound by applying a binder, such as a powder, paste, or melt, and melting the binder on the sheet or web of fibrous material 110.

The surface modification additive 120 is utilized to modify the surface of fibrous material 110 to lower the critical wetting surface tension and to prevent wound filler 100 from sticking to the wound. In embodiments, the surface modification additive imparts a critical wetting surface tension less than 50 dynes per centimeter, in some embodiments, less than 20 dynes per centimeter. The surface modification additive 120 may be a biocompatible chemical composition or melt which may be externally applied to fibrous material 110, as shown in FIG. 3A, or onto the individual fibers 112, as shown in FIG. 3B. Alternatively, the surface modification additive 120 may be internally applied to the fiber(s) 112 by extruding the additive with the fiber(s) during the manufacturing process. Exemplary surface modification additives include nonwoven hydrophobic repellent, such as PHOBOL® from Huntsman Chemical (Charlotte, N.C.), and melt additives from Goulston Technologies, Inc. (Monroe, N.C.).

The surface modification additive 120 may be externally or topically applied to fibrous material 110. In other embodiments, surface modification additive 120 may be applied to individual fiber(s) 112 prior to assembly of the fibrous material 110. Surface modification additive 120 may be applied by dipping, spraying, brushing, coating or otherwise finishing the exterior of the material with a chemical composition or melt. The chemical composition may be a dispersion, paste, emulsion, or other formulation containing waxes, silicones, fluorochemicals, and/or other hydrophobes within the purview of those skilled in the art suitable for lowering the critical wetting surface tension, and thus improving the nonadherency properties, of the material to which it is applied. The chemical composition may be cured or dried to form a wound filler 100 having an outer surface adapted to prevent adhesion of substances thereto and to allow fluids, such as exudates and gases, to pass therethrough. In other embodiments, a melt may be applied in a molten state and allowed to cool to form wound filler 100. The voids between the fiber(s) permit negative pressure applied to the reservoir to penetrate the wound “w”, and also permit exudates to be drawn through the fibrous material 110.

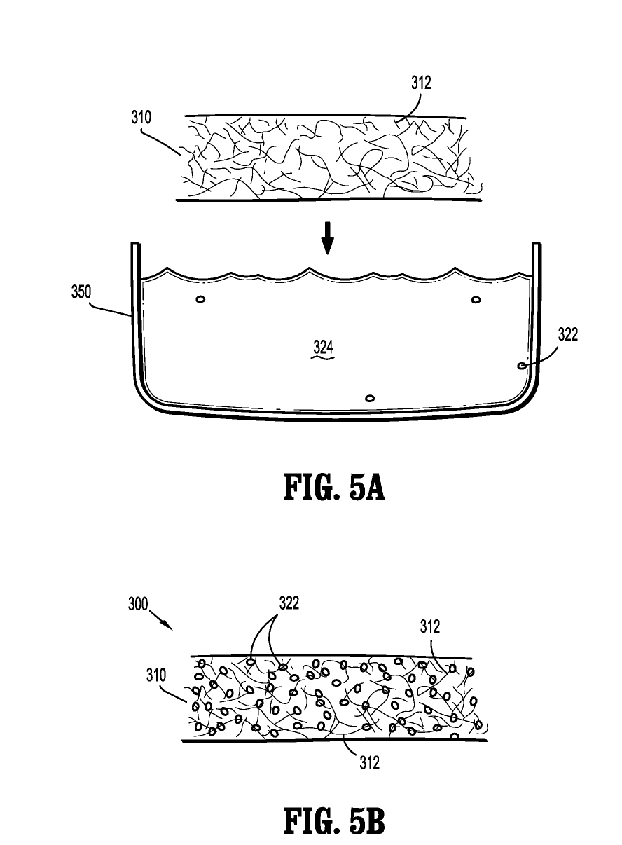

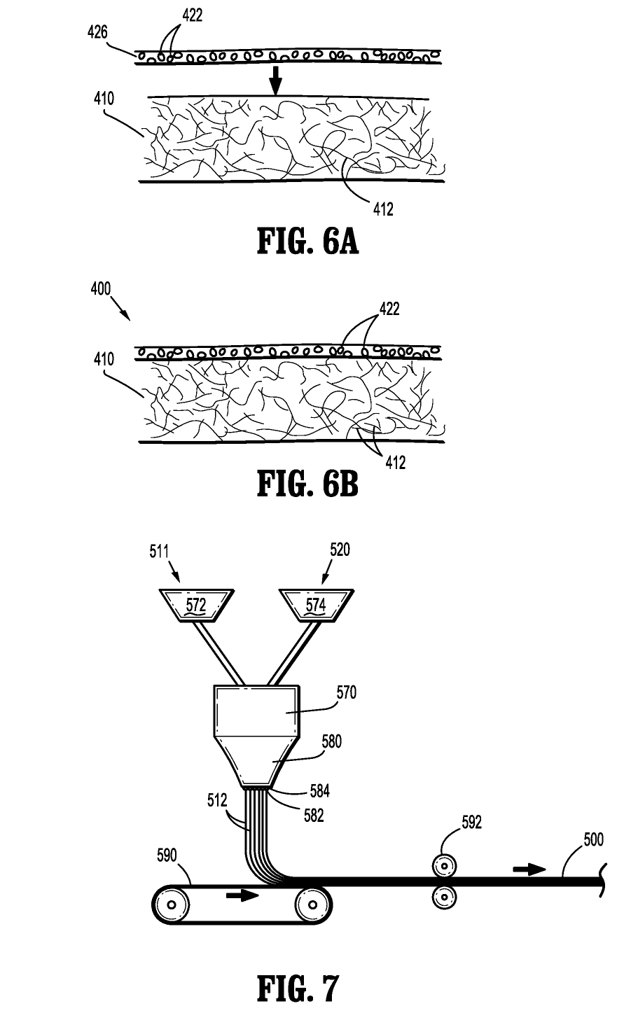

For illustrative purposes, FIGS. 4A and 4B show chemical composition 222 containing surface modification additive 220 being sprayed onto fibrous material 210 via dispenser or sprayer apparatus 260. Spraying chemical composition 222 onto the surface of fibrous material 210 coats the surface of fibrous material 210 thereby covering fibrous material 210 with surface modification additive 220. In FIGS. 5A and 5B, surface modification additive 220 is applied to fibrous material 310 via dipping. In FIG. 5A, the fibrous material 310 is shown facing a vessel 350 containing a solution 324 containing surface modification additive 320. The fibrous material 310 is submerged into the solution in vessel 350 by moving the fibrous material 310 in the direction of the arrows shown in FIG. 5A for a sufficient amount of time to allow solution 324 to coat fibrous material 310. Thereafter, as shown in FIG. 5B, the fibrous material 310 is removed from vessel 350 and is dried or cured to form a fibrous material 310 that is surface modified with additive 320. As illustrated in FIGS. 6A and 6B, the surface modification additive 420 may comprise a thin layer 426 of a hydrophobic material, such as a wax, polymeric film, or layer of melt adhesive, as described above. The layer 426 may be applied to fibrous material 410 and cured. The layer 426 containing the surface modification additive 420 may be perforated or apertured to permit negative pressure and exudates to transfer therethrough.

Alternatively, the individual fiber(s) 112 forming the fibrous material 110 may be added internally by extruding the surface modification additive 120 with the base polymeric resin that will be used to form fibers 112. In embodiments, a melt spinning process, such as spun bonding, melt blowing, and combinations thereof, may be utilized to form fiber(s) 112. Melt spinning processing equipment for forming fibers and nonwoven textiles are known and are commercially available.

An illustrative example of forming fibers containing surface modification additives via melt spinning is provided in FIG. 7. The raw polymer 511 used to form fibers 512 is fed into hopper 572 and the surface modification additive 520 is fed into hopper 574. The polymer 511 and additive 520 are added in sufficient proportion so that the additive will impart the desired characteristics to the fiber, i.e., the additive will bloom to the surface of the fiber to provide the desired properties thereto. The materials are fed into extruder 570 which melts and mixes the materials to form a homogenous melt. Each material may be separately heated before mixing. Extruder 570 then pressurizes and meters the melt in order to push it through spin pack 580. The melt is conveyed through shaped orifices or capillaries 582 on the face of the spinneret 584. The formed fibers 512 may be quenched, pressurized, or otherwise drawn to a desired fiber diameter and geometric shape configuration. Fibers 512 are passed onto a belt 590 or other surface where the fibers 512 can be collected. The collected fibers 512 may then be combined using mechanical, chemical, or thermal means, such as by entanglement, use of adhesives, heat, or pressure, as described above to form fibrous material 110. As illustrated in the present embodiment, the collected fibers 512 may be combined by hot calendering 592 to form nonwoven wound filler 500. Other thermal techniques may also be utilized to form the wound filler, such as belt calendering, through-air bonding, ultrasonic bonding, radiant heat bonding, and the like.

Any combination of methods as described above may be utilized to apply a surface modification additive onto a fibrous material or individual fiber(s) used to form the fibrous material. Further, the surface modification additive may be partially applied to the fibrous material, such as only on one side, or the fibrous material may be complete covered in the surface modification additive. In embodiments in which only a portion of the fibrous material is covered with a surface modification additive, such as a film applied only on one side, the fibrous material may be folded over onto itself such that the surface modified portion is the only portion of the fibrous material which contacts the wound bed.

The wound filler of the present disclosure may further be used for delivery of a bioactive agent. Thus, in some embodiments, at least one bioactive agent may be combined with the surface modification additive. The bioactive agent may be any substance or mixture of substances that have clinical use. Consequently, bioactive agents may or may not have pharmacological activity per se, e.g., a dye. Alternatively a bioactive agent could be any agent that provides a therapeutic or prophylactic effect, a compound that affects or participates in tissue growth, cell growth, cell differentiation, a compound that may be able to invoke a biological action such as an immune response, or could play any other role in one or more biological processes. It is envisioned that the bioactive agent may be applied to the wound filler in any suitable form of matter, e.g., films, powders, liquids, gels and the like.

Examples of classes of bioactive agents which may be utilized in accordance with the present disclosure include anti-adhesives, antimicrobials, antibacterials, antiobiotics, anti-virals, anti-fungals, anti-septics, anti-inflammatories, and anesthetics. It is also intended that combinations of bioactive agents may be used. For example, an anti-adhesive, to prevent adhesions from forming between the non-adherent gauze and the surrounding tissue, may be utilized with an antimicrobial, such as polyhexamethylene biguanide, to reduce the bio burden in the wound bed.

While the disclosure has been illustrated and described, it is not intended to be limited to the details shown, since various modifications and substitutions can be made without departing in any way from the spirit of the present disclosure. As such, further modifications and equivalents of the disclosure can occur to persons skilled in the art, and all such modifications and equivalents are intended to be within the spirit and scope of the disclosure as defined by the following claims.

What is claimed is:

1. An apparatus to promote the healing of an exuding wound comprising:
a wound cover for defining a reservoir over a wound in which a negative pressure may be maintained by forming a substantially fluid-tight seal around the wound;
a vacuum source in fluid communication with the reservoir, the vacuum source suitable for providing an appropriate negative pressure to the reservoir to stimulate healing of the wound; and
a wound filler positioned between the wound and the wound cover, the wound filler comprising a fibrous material treated with a surface modification additive.

2. The apparatus according to claim 1, wherein fibers of the fibrous material are individually treated with the surface modification additive.

3. The apparatus according to claim 1, wherein the surface modification additive is a melt adapted to coat the fibrous material to form a non-adherent coating thereon.

4. The apparatus according to claim 1, wherein the surface modification additive is a chemical composition adapted to coat the fibrous material to form a chemical finish thereon.

5. The apparatus according to claim 1, wherein the surface modification additive includes one of a wax, silicone, and fluorochemical.

6. The apparatus according to claim 1, wherein the fibrous material is treated with the surface modification additive by one of spraying, dipping, brushing, or melting.

7. The apparatus according to claim 1, wherein the fibrous material is treated with the surface modification additive by internally adding the surface modification additive to fibers of the fibrous material.

8. The apparatus according to claim 1, wherein the surface modification additive imparts a critical wetting surface tension less than 50 dynes per centimeter.

9. The apparatus according to claim 8, wherein the critical wetting surface tension is less than 20 dynes per centimeter.

10. The apparatus according to claim 1, wherein the fibrous material comprises a nonwoven material.

11. The apparatus according to claim 1, wherein the fibrous material comprises polypropylene.

12. The apparatus according to claim 1, wherein the surface modification additive includes a bioactive agent.

13. The apparatus according to claim 12, wherein the bioactive agent includes an antimicrobial.

14. A method of forming a surface modified fibrous material comprising the steps of:
applying a surface modification additive topically to a fibrous material; and
curing the additive to form a finish on the fibrous material.

15. The method of claim 14, wherein the step of applying a surface modification additive topically to a fibrous material further comprises applying the surface modification additive by one of spraying, dipping, brushing, and melt coating.

16. A method of forming a surface modified fibrous material comprising the steps of:
separately heating a raw polymer and a surface modification additive;
mixing the raw polymer and surface modification additive;
extruding homogenous fibers from the mixture; and
forming the fibers into a fibrous web.

17. The method of claim 16, wherein the step of forming the fibers into a fibrous web includes the step of thermally bonding the fibers together.
